(19) United States
(12) Patent Application Publication
(10) Pub. No.: US 2014/0187999 A1
(43) Pub. Date: Jul. 3, 2014

(54) APPARATUS, SYSTEM AND METHOD FOR PROVIDING IMAGE-GUIDED IN-VIVO BIOPSY WITH AT LEAST ONE CAPSULE

(71) Applicants: Guillermo J. TEARNEY, Cambridge, MA (US); Moon Gu LEE, Malden, MA (US); Sharath BHAGATUAL, New York, NY (US); Robert W. CARRUTH, Arlington, MA (US); Michalina GORA, Boston, MA (US)

(72) Inventors: Guillermo J. TEARNEY, Cambridge, MA (US); Moon Gu LEE, Malden, MA (US); Sharath BHAGATUAL, New York, NY (US); Robert W. CARRUTH, Arlington, MA (US); Michalina GORA, Boston, MA (US)

(73) Assignee: The General Hospital Corporation, Boston, MA (US)

(21) Appl. No.: 14/136,722

(22) Filed: Dec. 20, 2013

Related U.S. Application Data
(60) Provisional application No. 61/740,212, filed on Dec. 20, 2012.
(60) Provisional application No. 61/798,962, filed on Mar. 15, 2013.

(51) Int. Cl. A61B 10/04

(52) U.S. Cl. CPC A61B 10/04
USPC 600/563; 600/562

(57) ABSTRACT

An exemplary apparatus for obtaining a tissue sample(s) within a luminal anatomical structure can be provided. For example, an imaging first arrangement can be configured to generate an image of a portion(s) of the luminal anatomical structure, and a second arrangement can be configured to extract the tissue sample(s) based on the image. A pill-shaped housing can at least partially enclose the first and second arrangements.

### CROSS-REFERENCE TO RELATED APPLICATION(S)

This application relates to and claims priority from U.S. Patent Application Ser. No. 61/740,212 filed Dec. 20, 2012, and U.S. Patent Application Ser. No. 61/798,962 filed Mar. 15, 2013, the entire disclosures of which are incorporated herein by reference.

### FIELD OF THE DISCLOSURE

Exemplary embodiments of the present disclosure relate to apparatus, system and method which can utilize at least one capsule (or a single capsule which is to be swallowed) to, e.g., simultaneously image and retrieve samples of endo-luminal tissue, and to, e.g., provide image-guided in-vivo biopsy with at least one capsule.

### BACKGROUND INFORMATION

Diseases affecting the digestive tract (e.g., esophagus, stomach, small intestine, and large intestine) can be common, and may cause high morbidity and mortality. Many of these conditions, including cancer, eosinophilic esophagitis, H. Pylori gastritis, celiac disease, and inflammatory bowel disease cause observable damage to diseased tissue at the microscopic and macroscopic levels. To accurately diagnose and treat these conditions, tissue from the digestive tract often should be visualized and biopsied. Typically, this can be done by sedating the patient, guiding an endoscope into the digestive tract to visualize the surface of the diseased area, and using forceps to manually pull tissue away for collection. Such procedure is time-consuming, and makes patients uncomfortable, and can even make the patients gag, if not sedated. The sedation can sometimes be dangerous to the weak and the old. After the procedure, the patients may feel pain in their throat.

Recent advances in swallowed capsule endoscopy facilitate visualization of the entire digestive tract without need for sedation. In addition, capsules with optical frequency domain imaging (OFDI) technology allow visualization of subsurface pathology of endo-luminal tissue microscopically. However, these endoscopic and OFDI capsules and swallowed capsule are currently may not be able to biopsy tissue. It would be preferable to provide a single swallowed capsule to have the ability to both visualize tissue using endoscopic or OFDI diagnosis technology, and simultaneously obtain biopsies of this tissue for further testing.

Accordingly, there may be a need to address and/or overcome at least some of the issues and/or deficiencies described herein above.

### SUMMARY OF EXEMPLARY EMBODIMENTS

To address and/or overcome the above-described problems and/or deficiencies, exemplary embodiments of apparatus, system and method which can utilize at least one capsule (or a single capsule which is to be swallowed) to, e.g., simultaneously image and retrieve samples of endo-luminal tissue, and to, e.g., provide image-guided in-vivo biopsy with at least one capsule.

### Exemplary Image-Guidance of Swallowed Capsule Endoscope(s) to Pathology of Endo-Lumen

Certain exemplary embodiments of the apparati, methods and systems according to the present disclosure can (i) be image-guided by an imaging module, (ii) access to the target pathology, (iii) diagnose it macroscopically and/or microscopically, biopsies the tissue, and/or (iv) treat the lesion adequately in endo-luminal tract easily without sedation or acute danger to patients. The exemplary capsule(s) can be swallowed by the patients, and then moved by peristalsis motion of the patients' organ along their endo-lumen. The exemplary capsule(s) may be to stop and/or move actively by its own and/or via another mechanism mechanism.

Further, all or some surface of the endo-luminal tract can be monitored macroscopically by imaging camera and/or illuminating in the exemplary capsule(s) and/or diagnosed microscopically by OFDI image in the exemplary capsule(s). It is possible to diagnose the pathology, biopsy the sample tissue, dissect unwanted objects and/or treat lesion in the tract with, e.g., the aid of vision. For example, the image data can be transmitted to outside the patient's body using wired and/or wireless transmission. The exemplary capsule(s) can also have battery to power all electrics in it.

### Exemplary Collection of Endo-Luminal Tissue Samples While Imaging the Tissue

Certain exemplary embodiments of the methods and systems according to the present disclosure can be utilized, e.g., to image surface and subsurface features of endo-luminal tissue to identify abnormal tissue and specific regions of interest. Upon the identification of a tissue region of interest, this exemplary region can be biopsied and collected for further analysis.

Exemplary Excise of Unwanted Tissue or Foreign Objects from Digestive Tract

An exemplary embodiment of the system and method according to the present disclosure can be provided that can be used to visualize and/or remove unwanted objects and tissue, such as, e.g., precancerous or cancerous lesions, foreign objects, and parasites, from the digestive tract.

Exemplary Control of Capsule Movement within Digestive Tract

An exemplary embodiment of the system and method according to the present disclosure can be provided that can use exemplary mechanism(s) of the biopsy device to control the capsule within the endo-luminal tract while imaging tissue. For example, a clamp typically used to biopsy the tissue can attach the capsule to a region of interest for providing further detailed imaging. For example, extending and retracting component of the biopsy device/tool can apply force to control the capsule to a desired region and/or orientation within the endo-luminal tract.

### Exemplary Application of Spray Fluid for Improved Contrast or Targeted Therapy

An exemplary embodiment of the system and method according to the present disclosure can be used to spray a fluid from within the capsule onto the surrounding tissue. For example, the fluid can contain a contrast agent that facilitates higher quality imaging and/or therapeutics for a targeted treatment of the diseased tissue.

Many diseases of the digestive tract alter the tissue's microscopic and macroscopic properties. With exemplary embodiments of one or more image-guided biopsy capsule systems and methods according to the present disclosure, e.g., endo-luminal tissue surface and subsurface properties can be assessed, specific abnormal tissue or tissue regions of interest identified, and/or such tissue regions can be be biopsied and removed from the body for further testing. For example, unwanted foreign objects, parasites, and precancerous/cancerous tissue within the digestive tract can also be identified and removed using this capsule to prevent initiation and/or progression of disease. Advantages of these exemplary image-guided biopsy capsule methods and systems can include an ability to visualize the surface and the subsurface of endo-luminal tissue prior to biopsy or excision, and an ability to visualize and biopsy/excise the tissue without sedating the patient.

It can be challenging to control a capsule movement within the digestive tract. Therefore, it may be difficult to image and/or biopsy at or in certain endo-luminal areas. Accordingly to certain exemplary embodiments of the present disclosure, the exemplary mechanics of the biopsy device/tool can be provided to control the motion of the capsule. For example, an exemplary apparatus can be provided that can clamp the tissue to hold the capsule(s) in place; the operator can choose to excise the clamped tissue for the biopsy or release it to allow the capsule to continue its movement. Another embodiment is an apparatus that extends and retracts an arm from the capsule surface. This exemplary apparatus can extend the reach of the biopsy device/tool, and can also be used to apply force or torque to control the motion of the capsule(s).

Image quality can be enhanced and/or improved by spraying exemplary contrast agents on the imaged tissue. For example, acetic acid can be commonly used to localize and enhance Barrett's esophagus, a premalignant esophageal condition. According to an exemplary embodiment of the present disclosure, apparatus and method can be provided using which it is possible to spray the contrast agent to facilitate a higher quality imaging of the diseased tissue prior to biopsy. In another exemplary embodiment of the present disclosure, such exemplary apparatus and method can be used to release a therapeutic solution for a targeted treatment of the diseased tissue.

These and other objects of the present disclosure can be achieved by provision of an apparatus for obtaining a tissue sample(s) within a luminal anatomical structure which can include, for example, an imaging first arrangement which can be configured to generate an image of a portion(s) of the luminal anatomical structure, and a second arrangement which can be configured to extract the tissue sample(s) based on the image. A pill-shaped housing can partially enclose the first and second arrangements.

In some exemplary embodiments of the present disclosure, a tether can be attached to the housing; the tether can have a length that can be greater than 1 m, and a diameter that can be less than 3 mm. In some exemplary embodiments of the present disclosure, the image can be a video image. In certain exemplary embodiments of the present disclosure, an imaging third arrangement can generate a further image which can be different from the image, and can be of OCT, SECM, OFDI, confocal, 2 photon, 3 photon, flourenscence or Raman. The length of the housing can be less than 35 mm, and diameter of the housing can be less than 15 mm.

In certain exemplary embodiments of the present disclosure, the tether can include (i) a pullable cable or (ii) a pushable cable which can control an operation of the second arrangement. The second arrangement can include a forcep(s) which can be structured to grasp the tissue sample(s), and a collar arrangement which can be provided to compress a portion(s) of the forcep(s) so as to grasp the tissue sample(s). The second arrangement can include a (i) a pulley, (ii) a guide track, or (iii) cam mechanical linkages to translate a linear motion of a push and pull to operation of the forcep.

In some exemplary embodiments of the present disclosure, the pill shaped housing can incorporate a fluid delivery mechanism(s) for application of a contrast solution to a luminal organ tissue, which can include a (i) plunger, (ii) piston, (iii) spray nozzle, (iv) reservoir, or (v) tubing to control a flow of a fluid The fluid delivery mechanism can perform a (i) spray, (ii) a flush, (iii) a drain, or (iv) an aspiration. For example, the image can be a microscopic image, and the microscopic image can be generated by at least one of the following modalities: OCT, SECM, OFDI, confocal, 2 photon, 3 photon, fluorescence or Raman.

These and other objects, features and advantages of the exemplary embodiments of the present disclosure will become apparent upon reading the following detailed description of the exemplary embodiments of the present disclosure, when taken in conjunction with the appended paragraphs.

### BRIEF DESCRIPTION OF THE DRAWINGS

Further objects, features and advantages of the present disclosure will become apparent from the following detailed description taken in conjunction with the accompanying drawings showing illustrative embodiments of the present disclosure, in which:

FIG. 1A is an illustration of an exemplary embodiment of an exemplary optical frequency domain interferometry (OFDI) diagnosis, imaging, and biopsy capsule which includes endoscopic vision ability, microscopic comprehensive diagnosis ability, and/or biopsy devices, according to an exemplary embodiment of the present disclosure;

FIG. 1B is a schematic cross-sectional diagram of the exemplary embodiment of another image-guided biopsy capsule, containing OFDI diagnosis ability, endoscopic imaging ability, a biopsy device, and/or spray device according to another exemplary embodiment of the present disclosure;

FIG. 2A is a side view of a schematic diagram of a pulley system designed and/or configured to extend a biopsy device outward and clamp a tissue surrounding the capsule, according to still another exemplary embodiment of the present disclosure;

FIG. 2B is a front view of the schematic diagram of the pulley system shown in FIG. 2A;

FIG. 3A is a side view of a schematic diagram of the exemplary pulley system shown in FIGS. 2A and 2B in an initial configuration which can perform, e.g., an “extend-clamp” part of an exemplary “extend-clamp-retract” tissue biopsy mechanism, according to an exemplary embodiment of the present disclosure;

FIG. 3B is a from view of the schematic diagram of the pulley system shown in FIG. 3A;

FIG. 3C is a side view of a schematic diagram of the exemplary pulley system shown in FIG. 3A in an extended configuration, according to an exemplary embodiment of the present disclosure;

FIG. 3D is a front view of the schematic diagram of the pulley system shown in FIG. 3C;

FIG. 3E is a side view of a schematic diagram of the exemplary pulley system shown in FIGS. 3A and 3C in a clamped configuration, according to an exemplary embodiment of the present disclosure;

FIG. 3F is a front view of the schematic diagram of the pulley system shown in FIG. 3E;

FIG. 4A is a side view of a schematic diagram of a pulley system designed and/or configured to designed to retract the biopsy device after it has clamped tissue, according to yet another exemplary embodiment of the present disclosure;

FIG. 4B is a front view of the schematic diagram of the pulley system shown in FIG. 4A;

FIG. 5A is a side view of a schematic diagram of the exemplary pulley system shown in FIGS. 4A and 4B in an initial configuration which can perform, e.g., an “clamp” part of the exemplary “extend-clamp-retract” tissue biopsy mechanism, according to an exemplary embodiment of the present disclosure;

FIG. 5B is a front view of the schematic diagram of the pulley system shown in FIG. 5A;

FIG. 5C is a side view of a schematic diagram of the exemplary pulley system shown in FIG. 5A in an retracted configuration, according to an exemplary embodiment of the present disclosure;

FIG. 5D is a front view of the schematic diagram of the pulley system shown in FIG. 3C;

FIG. 6A is an exemplary image of the exemplary tissue biopsy tool/device within the OFDI diagnosis capsule according to an exemplary embodiment of the present disclosure, in an initial configuration;

FIG. 6B is an exemplary image of the exemplary tissue biopsy tool/device of FIG. 6A, providing an extended cutter arrangement;

FIG. 6C is an exemplary image of the exemplary tissue biopsy tool/device of FIG. 6B, using which the tissue is clamped and cut;

FIG. 6D is an exemplary image of the exemplary tissue biopsy tool/device of FIG. 6C, in a retracted configuration;

FIG. 7A is an illustration of a tethered capsule arrangement according to yet another exemplary embodiment of the present disclosure which provides biopsy and OFDI diagnosis, in an initial configuration;

FIG. 7B is an illustration of the tethered capsule arrangement or FIG. 7A, in an extended configuration;

FIG. 7C is an illustration of the tethered capsule arrangement of FIG. 7A, in a clamp configuration;

FIG. 7D is an illustration of the tethered capsule arrangement of FIG. 7A, in a retracted configuration;

FIG. 8 is a schematic side cross-sectional diagram of a wireless capsule apparatus with biopsy, spray, endoscopic imaging, and OFDI diagnosis capabilities, according to still another exemplary embodiment of the present disclosure;

FIG. 9 is a schematic side cross-sectional diagram of a suction and guillotine mechanism for obtaining a biopsy, according to yet another exemplary embodiment of the present disclosure; and

FIG. 10 is a schematic perspective diagram of an imaging module and OFDI diagnosis capabilities, according to still further exemplary embodiments of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION AND EXEMPLARY DATA

According to an exemplary embodiment of the present disclosure, it is possible to utilize an image-guided biopsy capsule to localize and biopsy tissue of interest with high spatial accuracy and without need for a patient sedation, as follows.

### Exemplary Development and Validation of Exemplary Image-Guided Biopsy Capsule System and Method

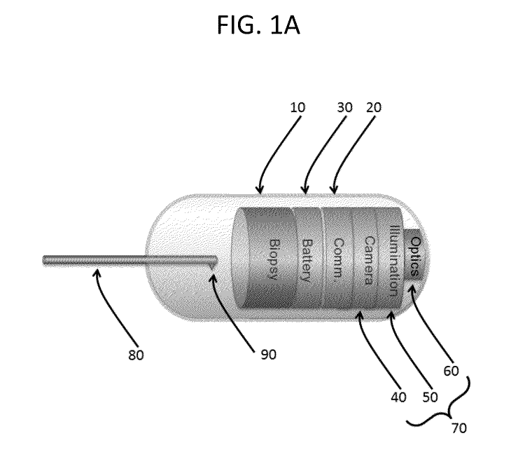

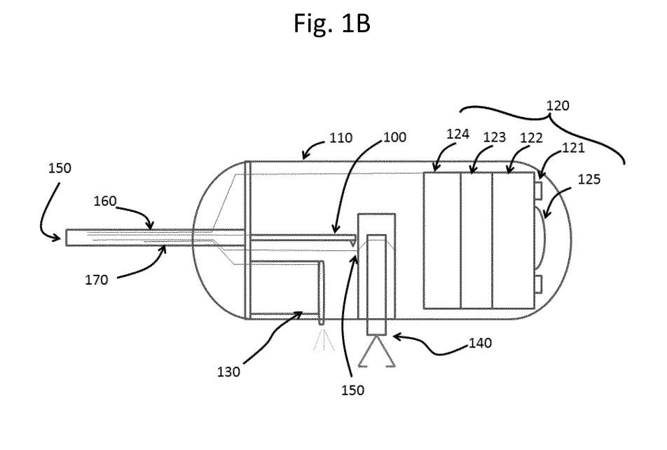

As shown in FIG. 1A, an exemplary embodiment of an imaging and biopsy capsule can include a capsule having a biopsy module 10, an imaging module 70, and a microscopic/comprehensive diagnosis module, such as, e.g., an OFDI probe 90. The imaging module 70 can include a vision camera (e.g. CCD or CMOS) 40, illuminating arrangement (e.g., LED or bulb) 50, and optics arrangement 60. The illumination arrangement 50 can include an illuminating structure which can be configured to brighten the vision in gastrointestinal (GI) tract surface. The optics arrangement 60 can be provided to widen field of view of the GI tract surface or to focus on the pathological area. The exemplary OFDI probe 90 can diagnose the organ microscopically over wide range comprehensively using its image data. The biopsy can sample the pathology tissue from the organ. The image and OFDI data may be transmitted to outside receiver by a signal line. The exemplary camera 40 and the illumination arrangement 50 can be powered by electric wire form outside power supply. The biopsy can be operated by a tether. The electric wires and tether can be provided in a sheath and/or micro-lumen tube 80. Instead or (or in an addition to) the wired data transmission and power delivery for imaging module, the exemplary capsule can include a wireless communication module 20 and/or a battery for power 30. The exemplary communication module 20 may transmit the image data to a receiver outside the body. The battery 30 or a plurality of batteries can supply power to the electronics arrangement, such as the camera 40, the illumination arrangement 50, the communication module 20, etc. This exemplary embodiment which can provide a wireless communication can be more comfortable for patients, simplify the device(s), and/or help to reduce the size of or miniaturize the exemplary capsule.

FIG. 1B shows another exemplary embodiment of the image-guided biopsy capsule can be provided which facilitates a patient to swallow an exemplary capsule containing an on-board imaging module, biopsy, and fluid spray devices. As shown in FIG. 1B, this exemplar capsule can use an OFDI probe 100 to image subsurface features of the tissue surrounding the capsule body 110. The imaging module can have an illumination arrangement 121, a camera 122, a communication arrangement 123, a battery 124, and/or an optics arrangement 125 to obtain and/or transmit vision of the surrounding tissue. The exemplary capsule can include a spray device 130 which can be configured to spray the region with a contrast agent that can enhance visualization or a therapeutic solution that delivers targeted treatment to the lesion. Finally, the exemplary capsule can include a device 140 to biopsy tissue after visual inspection and localization. The exemplary capsule can be attached to a tether 150 that facilitates a direct (or indirect) contact and communication with the operator. The tether 150 can include conducting wire(s) 160 configured to transmit imaging data in real-time, and flexible wire(s) 170 configured to facilitate a manual control of the biopsy and spray devices. Another conducting wire can also be used to transmit power to the onboard imaging components. According to one exemplary embodiment of the present disclosure, the electrical wire(s) for transmitting data and power delivery can be excluded when utilizing the communication arrangement (e.g., module) 123 and the battery 124, as shown in FIG. 1B.

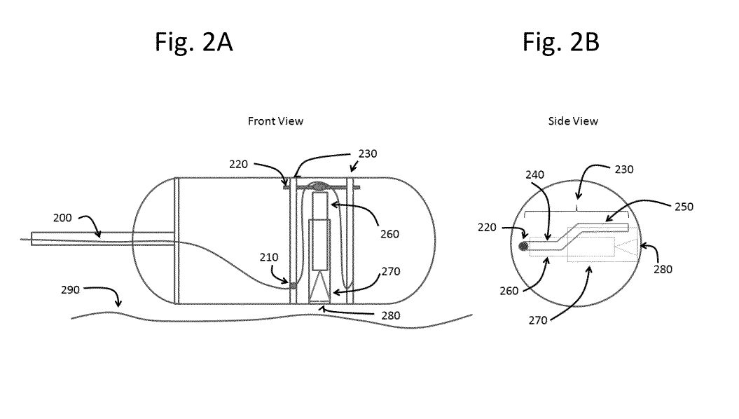

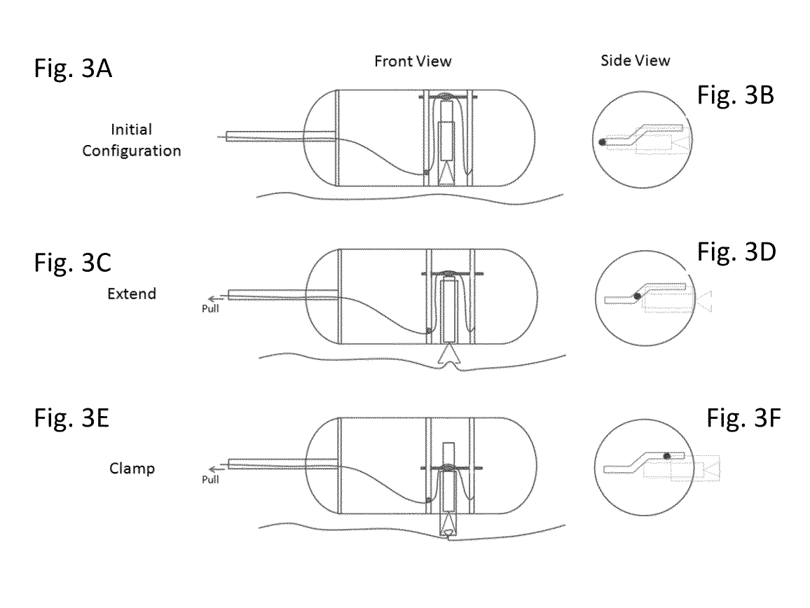

In such exemplary embodiment, as shown in FIGS. 2A-5D and 7, the exemplary biopsy device(s) can use an “extend-clamp-retract” mechanism to obtain biopsy samples of the tissue. This exemplary mechanism can be driven by, e.g., two or more mechanical pulley systems. For example, a first pulley system (shown in FIGS. 2A-3F) which is responsible for extending and clamping the tissue, can include a flexible wire 200, a fixed pulley wheel 210, a moving, pulley axle 220 that pushes the biopsy apparatus, and a system of rails 230 that is configured to guide the axle 220 as the flexible wire 200 is pulled. The operator can pull on the flexible wire 200, thus causing the axle to move along one or more of the rail 230, e.g., perpendicular to the direction the wire 200 is pulled. As the operator pulls the wire 200, the axle 220 can be initially guided along a first rail 240, and can force and/or facilitate a biopsy clamping tool 260 to extend outward to contact a surrounding tissue 290. As the operator continues to pull the wire 200, the axle 220 can be guided onto a second rail 250. While moving along this second rail 250, the axle 220 can force and/or facilitate an outer sheath 270 to extend outward over the biopsy clamping tool 260. As an outer sheath extends over the clamping tool 260, it can force the teeth of a clamp 280 to shut with the tissue 290 trapped therein. An exemplary “extend-clamp” function of the first pulley system is illustrated in FIGS. 3A-3F.

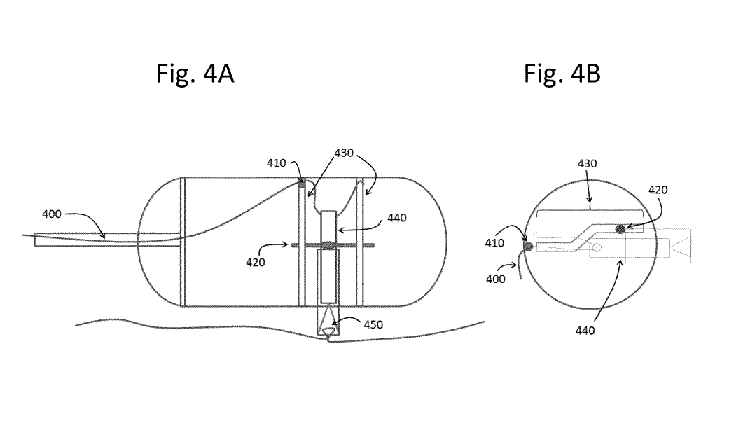

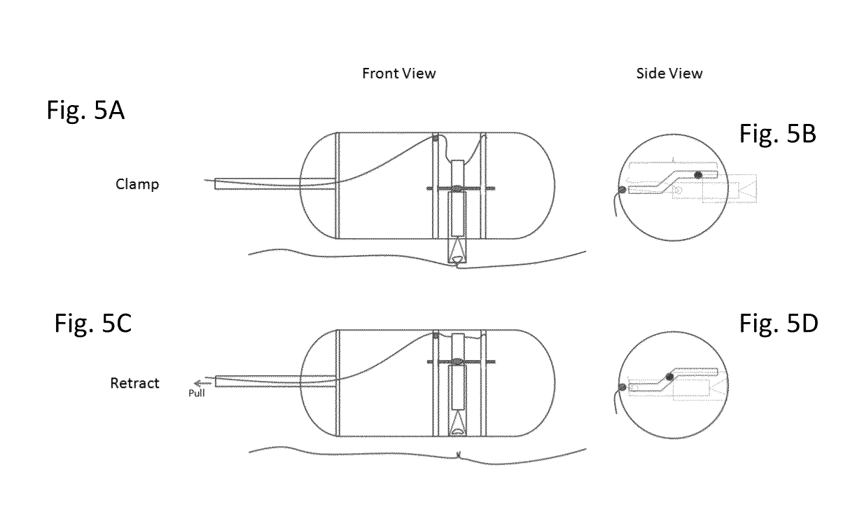

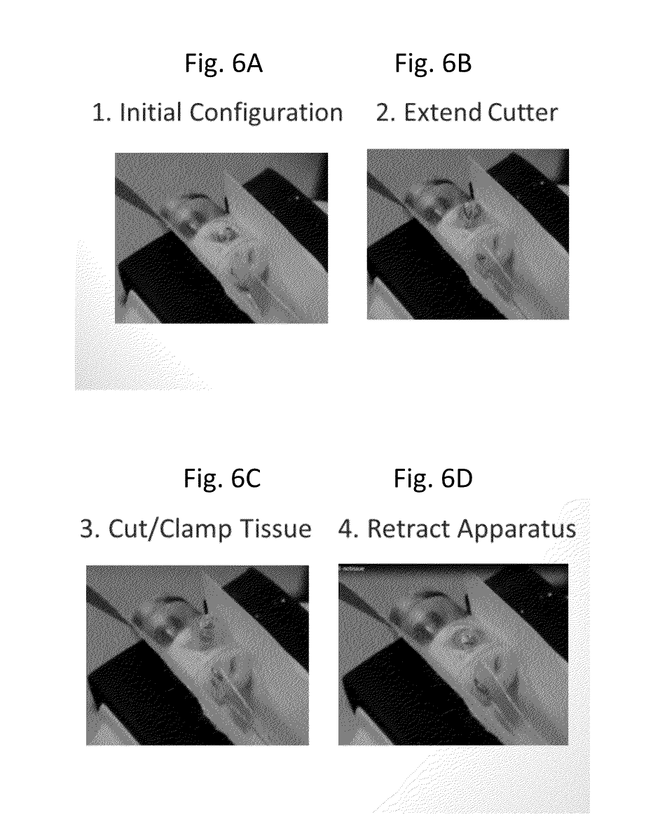

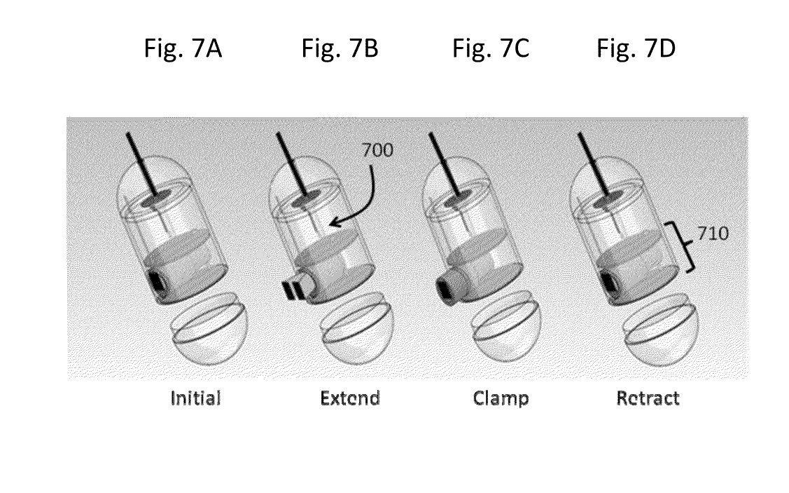

A second pulley system, as shown in FIGS. 4A-5D, can include a second flexible wire 400, a second fixed pulley wheel 410, the same moving pulley axle 420 and another system of rails 430 which can be used to retract the biopsy apparatus. For example, the operator can pull on the second flexible wire, which can be threaded to a back portion of a biopsy apparatus 440 (as shown in FIGS. 4A and 4B) or threaded around the movable axle 420. As the operator pulls on the second wire, the entire biopsy apparatus, including, e.g., biopsy clamping tool, outer sheath, and trapped tissue 450, can be retracted back into the exemplary capsule (as shown in FIGS. 5A-5D). The retraction force can result in the tissue 450 being ripped away from the endoluminal surface. The tissue 450 can be trapped within the exemplary capsule, e.g., inside the biopsy apparatus. This exemplary “extend-clamp-retract” biopsy mechanism has been implemented as shown in FIGS. 6A-7D), and has been shown to effectively grasp and the biopsy tissue ex vivo.

Exemplary embodiments of the image-guided biopsy capsule according to the present disclosure can contain one or more of the devices discussed (imaging module 120, OFDI probe 100, biopsy device 140, and spray device 130). According to one exemplary embodiment of the present disclosure, as shown in FIGS. 7A-7D), an exemplary capsule can be provided with only an OFDI probe 700 and a biopsy device 710. In contrast, the exemplary embodiment described herein above and illustrated in FIG. 1 contains a capsule combining, e.g., all of the components discussed. Other exemplary embodiments can contain different combinations of imaging, biopsy, and spray mechanisms tailored to each individual patient.

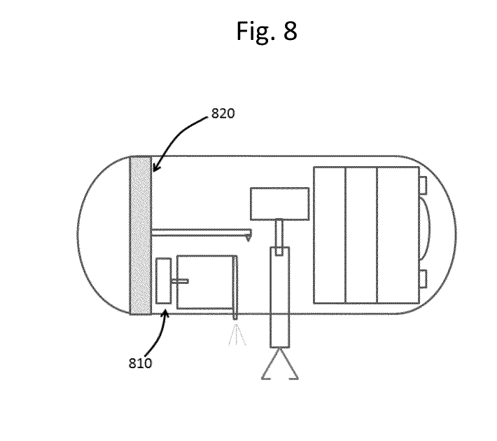

In certain exemplary embodiments of the present disclosure, the exemplary capsule can be wired, or may also be wireless (as shown in FIG. 8). In the embodiments having a wireless capability/configuration, a wireless transmitter can be used to transmit imaging data to the operator, and onboard motor(s) 810 can be used to control the biopsy and spray devices. An onboard microcontroller 820 also be used to control these motor(s) and OFDI modules/devices. Onboard batteries can be used to power the motors, microcontroller, and imaging devices.

According to further exemplary embodiments of the present disclosure, the exemplary biopsy device can include an extending/retracting arrangement (e.g., one or more arms) that can be moved out and/or away from the capsule body. This exemplary arm arrangement can be controlled with, e.g., a pulley system, a spring, onboard motors, or a combination of these three mechanisms. Such exemplary arm arrangement can be used to force the biopsy device into contact with surrounding tissue, and/or to control capsule movement. In still other exemplary embodiments, the same pulley system used to extend and retract the biopsy apparatus can also be used to control capsule motion to desired regions or orientation in the digestive tract. The “extend-clamp-retract” mechanism used to biopsy the tissue can also be used to temporarily grasp and release the tissue as a procedure for controlling the capsule movement.

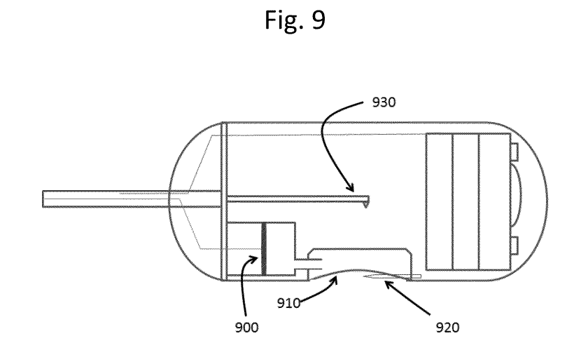

In yet further exemplary embodiments of the present disclosure, as shown in FIG. 9, the biopsy device can include a plunger mechanism 900 to generate a suction 910 and draw tissue into the capsule. This exemplary plunger can be provided onboard of the exemplary capsule, and can be controlled with a pulley, a spring, onboard motors, or a combination of these three mechanisms. The trapped tissue can be cut using a guillotine blade 920. The plunger/suction mechanism can also be used to draw tissue to an appropriate working distance for, e.g., an optimal imaging with an OFDI probe 930. Suction and release function(s) of the tissue may also serve as a procedure for controlling the capsule movement.

As shown in FIG. 8, further exemplary embodiments of the capsule can be provided that can contain the spray mechanism which can use a motor 810, a wire within a tether 170, a pulley system, a spring, or a combination of these methods to force fluid out of the capsule. The spray device may release a contrast agent for a better viewing of the tissue. It may also potentially release therapeutics for a targeted drug delivery following imaging and localization of the abnormal tissue. The fluids intended for spray can be stored on a container within the exemplary capsule and/or fed through a tether from outside the patient's body.

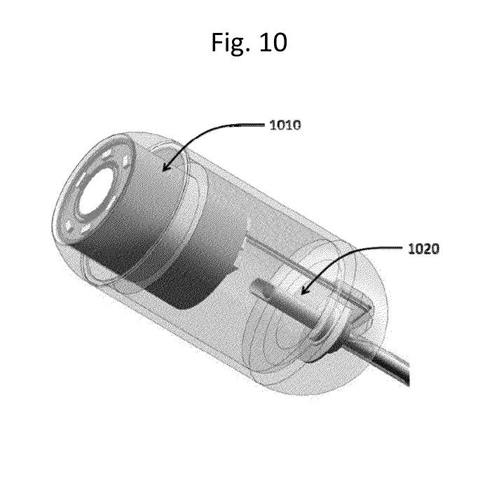

FIG. 10 shows yet additional exemplary embodiments of the endoscopic capsule according to the present disclosure which can include imaging module and OFDI diagnosis. As an example, the exemplary capsule can include only an OFDI probe 1020 and a macroscopic imaging module 1010. The imaging module 1010 can assist in navigating the capsule to the pathology in the endo-lumen and the OFDI diagnoses pathological lesion.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in international Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

What is claimed:

1. An apparatus for obtaining at least one tissue sample within a luminal anatomical structure, comprising:
an imaging first arrangement which is configured to generate an image of at least one portion of the luminal anatomical structure;
a second arrangement which is configured to extract the at least one tissue sample based on the image; and
a pill-shaped housing at least partially enclosing the first and second arrangements.

2. The apparatus according to paragraph 1, further comprising a tether attached to the housing.

3. The apparatus according to paragraph 2, wherein a length of the tether is greater than 1 m.

4. The apparatus according to paragraph 2, wherein a diameter of the tether is less than 3 mm.

5. The apparatus according to paragraph 1, wherein the image is a video image.

6. The apparatus according to paragraph 2, comprising an imaging third arrangement which generates a further image which is different from the image, and is at least one of OCT, SECM, OFDI, confocal, 2 photon, 3 photon, flourenscence or Raman.

7. The apparatus according to paragraph 1, wherein a length of the housing is less than 35 mm, and diameter of the housing is less than 15 mm.

8. The apparatus according to paragraph 1, wherein the image is at least one of OCT, SECM, OFDI, confocal, 2 photon, 3 photon, florenscence or Raman.

9. The apparatus according to paragraph 2, wherein the tether includes at least one of (i) a pullable cable or (ii) a pushable cable which controls an operation of the second arrangement.

10. The apparatus according to paragraph 1, wherein the second arrangement includes at least one forcep which is structured to grasp the at least one tissue sample, and a collar arrangement which is provided to compress at least one portion of the at least one forcep so as to grasp the at least one tissue sample.

11. The apparatus according to paragraph 10, wherein the second arrangement includes at least one of (i) a pulley, (ii) a guide track, or (iii) cam mechanical linkages to translate a linear motion of a push and pull to operation of the forcep.

12. The apparatus according to paragraph 1, wherein the pill shaped housing incorporates at least one fluid delivery mechanism for application of a contrast solution to a luminal organ tissue comprising at least one of (i) plunger, (ii) piston, (iii) spray nozzle, (iv) reservoir, or (v) tubing to control a flow of a fluid.

13. The apparatus according to paragraph 12, wherein the fluid delivery mechanism performs at least one of (i) sprays, (ii) flushes, (iii) drains, or (iv) aspirates.

14. The apparatus according to paragraph 1, wherein the image is a microscopic image.

15. The apparatus according to paragraph 14, wherein the microscopic image is generated by at least one of the following modalities: OCT, SECM, OFDI, confocal, 2 photon, 3 photon, fluorescence or Raman.

16. A method for obtaining at least one tissue sample within a luminal anatomical structure, comprising:
generating an image of at least one portion of the luminal anatomical structure with an imaging first arrangement; and
extracting the at least one tissue sample based on the image with a second arrangement, wherein the first and second arrangements are at least partially enclosed by a pill-shaped housing.
